(19) United States
(12) Patent Application Publication
(10) Pub. No.: US 2021/0379025 A1
(43) Pub. Date: Dec. 9, 2021

(54) COMPOSITIONS AND METHODS FOR BLOCKING THE CYTOKINE STORM OF CORONAVIRUS AND OTHER MEDICAL CONDITIONS

(71) Applicant: Thomas Winston, Stillwell, KS (US)

(72) Inventor: Thomas Winston, Stillwell, KS (US)

(21) Appl. No.: 16/946,105

(22) Filed: Jun. 5, 2020

(51) Int. Cl. A61K 31/436; A61K 31/155; A61K 31/232; A61K 31/05; A61K 45/06

(52) U.S. Cl. CPC A61K 31/436; A61K 31/155; A61K 45/06; A61K 31/05; A61K 31/232

(57) ABSTRACT

Disclosed herein are pharmaceutical compositions for the treatment of cytokine storm syndrome, and methods of administering such compositions for the treatment of acute respiratory distress syndrome, and the prevention of multiple organ failure in patients with cytokine storm syndrome and/or acute respiratory distress syndrome, comprising an effective amount of one or more mTor inhibitors.

### FIELD

The field of the disclosure relates generally to compositions for the treatment of cytokine storm syndromes. More specifically, the field of disclosure relates generally to compositions for the treatment of cytokine storm syndromes that include mTor inhibitors. More specifically, the field of disclosure relates to blocking the cytokine storm resulting from infection by pathogens such as coronavirus and including COVID-19.

### BACKGROUND

Cytokine Storm Syndromes (CS S) are a group of disorders characterized by a cascade of exaggerated immune responses. In people experiencing CSS, certain cytokines are present in the blood at abnormally high levels. CSS can cause a variety of symptoms, ranging from mild, flu-like symptoms to more severe symptoms such as hypotension and blood clots. CSS can affect multiple organ systems, leading to multiple organ dysfunction, multiple organ failure, and death.

CSS may be triggered by several different types of underlying medical issues, including genetic syndromes, infection, cancer, and autoimmune diseases. CSS may also be a side effect of certain medical treatments, such as immunotherapy, and organ or stem cell transplant. Currently, many of the deaths due to COVID-19 are a result of CSS.

Currently, CSS is treated primarily by supportive measures such as, intensive monitoring of vital signs, ventilator support, IV fluids, electrolyte management and hemodialysis. No current drug treatments are currently available to treat CSS. There remains a need for an effective drug treatment for CSS to prevent serious complications such as multiple organ dysfunction syndrome, multiple organ failure, and death. What is further needed is a treatment for the novel coronavirus COVID-19 that would block or reduce the severity of CSS resulting from infection by such a virus.

### BRIEF DESCRIPTION

Disclosed herein is a pharmaceutical composition for the treatment of cytokine storm syndrome including an effective amount of one or more mTor inhibitors.

In another aspect, disclosed herein is a method of treating cytokine storm syndrome in a patient, including administering an effective amount of one or more mTor inhibitors.

In another aspect, disclosed herein is a method of treating acute respiratory distress syndrome in a patient, such as occurs in patients infected with coronavirus including COVID-19, including administering an effective amount of one or more mTor inhibitors.

In another aspect, disclosed herein is a method of preventing multiple organ failure in a patient with cytokine storm syndrome, including administering an effective amount of one or more mTor inhibitors.

### DETAILED DESCRIPTION

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or a circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged; such ranges are identified and include all the sub-ranges contained therein unless context or language indicates otherwise.

As used herein, the term “patient” refers to a warm blooded animal such as a mammal which is the subject of a medical treatment for a medical condition that causes at least one symptom. It is understood that at least humans, dogs, cats, and horses are within the scope of the meaning of the term. In some aspects, the patient is human. Generally, as used herein, the term “patient” means a human or an animal for which the composition of the disclosure may be administered.

As used herein, the term “cytokine storm syndrome (CSS)” refers to any disorder characterized by a severe immune reaction in which the body releases too many cytokines into the blood too quickly.

As used herein, the term “acute respiratory distress syndrome (ARDS)” refers to severe, acute lung dysfunction affecting all or most of both lungs of a patient that occurs as a result of illness or injury.

As used herein, the term “multiple organ failure (MOF)” refers to the failure of two or more systems in the body, such as the cardiovascular and renal systems. MOF may be induced by CSS.

As used herein, the term “reduce the incidence of” refers to a reduction in the number of clinical signs of CSS and/or ARDS in an individual subject or in a group of subjects when comparing an individual subject or group of subjects that have received an administration of a composition of the disclosure and an individual subject or group of subjects that have not received an administration of a composition of the disclosure.

As used herein, the term “reduce the severity of” refers to a reduction in the severity of clinical signs of CSS and/or ARDS in an individual subject or in a group of subjects when comparing an individual subject or group of subjects that have received an administration of a composition of the disclosure and an individual subject or group of subjects that have not received an administration of a composition of the disclosure.

In various embodiments, the compositions of the disclosure include compositions for the treatment of CSS and ARDS. In various embodiments the compositions of the disclosure further include compositions for the prevention of multiple organ failure in a patient with CSS and/or ARDS. In various embodiments, the compositions of the disclosure include an effective amount of one or more mTor inhibitors.

Without being bound by theory, it is believed that mTor complex 1 and complex 2 control multiple diffuse aspects of cellular metabolism and cellular death. It is believed that the gene complex activity is enhanced and driven upwards by cytokine release. mTor complex 1 and complex 2 may be down regulated by the use of mTor inhibitors. In various embodiments, suitable mTor inhibitors may include non-antibiotic macrolides, biguanide antihyperglycemic agents, omega-3 fatty acid derivatives, ATP-competitive mTor kinase inhibitors, and other agents that effectively inhibit mTor protein kinase.

In various embodiments, the compositions of the disclosure may include an effective amount of a non-antibiotic macrolide. Suitable non-antibiotic macrolides include rapamycin and rapamycin derivatives, such as temsirolimus, everolimus, ridaforolimus, and the like. In various embodiments, the compositions of the disclosure include an effective amount of at least about 0.5 mg of a non-macrolide antibiotic, or between about 0.5 mg to about 40 mg, or 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 mg or any range between any two of these amounts including between about 3 to about 30 mg, and between about 6 mg to about 40 mg.

In various embodiments, the compositions of the disclosure may include an effective amount of a biguanide antihyperglycemic agent. Suitable biguanide antihyperglycemic agents include metformin. In various embodiments, the composition may include an effective amount of at least about 50 mg of biguanide antihyperglycemic agent, or between about 50 to about 3000 mg, or 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, and 3000 mg or any range between any two of these amounts including about 250 mg to about 3000 mg, about 250 mg to about 500 mg, about 250 mg to about 750 mg, 250 mg to about 1000 mg, about 250 mg to about 1250 mg, or between about 250 mg to about 1500 mg.

In various embodiments, the composition may include an effective amount of an omega-3 fatty acid derivative. Suitable omega-3 fatty acid derivatives may include icosapent ethyl. In various embodiments, the composition of the disclosure may include an effective amount of at least about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 g of icosapent ethyl, or about 0.5 g to 6 g of icosapent ethyl, or about 1 g to about 3 g icosapent ethyl, or about 2 g to 4 g of icosapent ethyl, or about 3 g to 6 g of icosapent ethyl. In some preferred forms, the amount of icosapent ethyl is sufficient to maintain an optimum level of icosapent ethyl in the blood of a subject receiving an administration of the composition.

In various embodiments, the composition may include an effective amount of resveratrol. In various embodiments, the composition may include an effective amount of at least about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 mg of resveratrol, or from 100 mg to about 1000 mg of resveratrol.

In various embodiments, the composition may further include at least one additional active ingredient. Suitable additional active ingredients include effective amounts of a steroid, glucocorticoid or other anti-inflammatory corticosteroid. Suitable steroids include 21-acetoxypregnenolone, alclometasone, algestone, anacortave acetate, amcinonide, beclomethasone, betamethasone, budesonide, chloroprednisone, clobetasol, clobetasone, clocortolone, cloprednol, cortieosterone, cortisone, cortivazol, deflazacort, desonide, desoximetasone, diflorasone, diflucortolone, difuprednate, enoxolone, fluazacort, flucloronide, flumethasone, flunisolide, fluocinolone acetonide, fluocinonide, flucloronide, flumethasone, flunisolide, fluocortin butyl, fluocortolone, fluorometholone, fluperolone acetate, flupreduisolone, fluraudrenolide, fluticasone propionate, hydrocortamate, hydrocortisone, meprednisone, methylpreduisolone, paramethasone, prednisolone, prednisolone 21-diethylaminoacetate, fluprednidene acetate, formocortal, loteprednol etabonate, medrysone, mometasone furoate, prednicarbate, prednisolone, prednisolone 25-diethyiaminoacetate, prednisolone sodium phosphate, prednisone, prednival, prednylidene, triamcinolone, triamcinolone acetonide, triamcinolone benetonide, and triamcinolone hexacetonide, and analogs, derivatives, pharmaceutically acceptable salts, esters, prodrugs, codrugs, and protected forms thereof. Suitable steroids include halcinonide, halbetasol propionate, halometasone, halopredone acetate, isoflupredone, loteprednol etabonate, niazipredone, rimexoione, and tixocortol, and analogs, derivatives, pharmaceutically acceptable salts, esters, prodrugs, co-drugs, and protected forms thereof.

In various embodiments, the composition may include an effective amount of at least about 10 mg to about 1500 mg of a steroid. In various embodiments the composition may include an effective amount of at least about 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075 1100, 1125, 1150, 1175, 1200, 1225, 1250, 1275, 1300, 1325, 1350, 1375, 1400, 1425, 1450, 1475, and 1500 mg of a steroid, or any range between any two of these amounts including from about 125 mg to about 1000 mg.

In various embodiments, the composition of the disclosure may further contain additional pharmaceutically acceptable carriers. The pharmaceutical composition may, for example, be in a form suitable for oral administration as a tablet, capsule, pill, powder, sustained release formulation, solution or suspension, in a form suitable for parenteral injection as a sterile solution, suspension, or in a form of an emulsion for topical administration as an ointment or cream or for rectal administration as a suppository. The pharmaceutical composition may be in unit dosage forms suitable for single administration of precise dosages. The pharmaceutical composition may include conventional pharmaceutical carriers or excipients. In addition, the compositions may include other medicinal or pharmaceutical agents, carriers, adjuvants, etc.

In various embodiments, the composition may be administered to a patient through any suitable route of administration effective in delivering an amount of active agent or active agents to a patient. Suitable routes of administration include oral, parenteral, enteral, rectal or the like.

Various embodiments of the disclosure further relate to methods of treating CSS and ARDS and/or preventing multiple organ failure in patients with CSS and/or ARDS and/or reducing the incidence and/or severity of CSS and/or ARDS that include administering an effective amount of one or more mTor inhibitors. In various embodiments, suitable mTor inhibitors may include non-antibiotic macrolides, biguanide antihyperglycemic agents, omega-3 fatty acid derivatives, ATP-competitive mTor kinase inhibitors, and other agents that effectively inhibit mTor protein kinase.

In various embodiments, the methods may include administering an effective amount of a non-antibiotic macrolide. Suitable non-antibiotic macrolides include rapamycin and rapamycin derivatives, such as temsirolimus, everolimus, ridaforolimus, and the like. In various embodiments, the methods may include administering an effective amount of at least about 0.5 mg of a non-antibiotic macrolide, or between about 0.5 mg to about 40 mg, or 0.5 mg to about 40 mg, or 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40 mg or any range between any two of these amounts including between about 3 to about 30 mg, and between about 6 mg to about 40 mg per day. In various embodiments, the non-antibiotic macrolide may first be administered as a loading dose of about 3 mg/m² followed by a maintenance dose of about 1 mg/m² once daily.

In various embodiments, the methods may include administering an effective amount of a biguanide antihyperglycemic agent. Suitable biguanide antihyperglycemic agents include metformin. In various embodiments, the methods may include administering an effective amount of at least about 250 mg of metformin or at least about 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, or 1500 mg, or about 250 mg to about 1500 mg once, twice, or three or more times daily.

In various embodiments, the methods may include administering an effective amount of an omega-3 fatty acid derivative. In various embodiments, the omega-3 fatty acid derivative may be administered at a dose of at least about 0.5, 0.6, 0.7, 0.8, 0.9, 1.0, 1.1, 1.2, 1.3, 1.4, 1.5, 1.6, 1.7, 1.8, 1.9 g, or about 2 grams once, twice, or three or more times a day.

In various embodiments, the methods may include administering an effective amount of resveratrol. In various embodiments, resveratrol may be administered at a dose of at least about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 mg, or about 100 mg to about 300 mg daily. This dose can be divided among 1, 2, 3, or more doses.

In various embodiments, the methods may further include administering an effective amount of an additional active ingredient. Suitable additional active ingredients include effective amounts of a steroid, glucocorticoid or other anti-inflammatory corticosteroid. Suitable steroids include 21-acetoxypregnenolone, alclometasone, algestone, anacortave acetate, amcinonide, beclomethasone, betamethasone, budesonide, chloroprednisone, clobetasol, clobetasone, clocortolone, cloprednol, cortieosterone, cortisone, cortivazol, deflazacort, desonide, desoximetasone, diflorasone, diflucortolone, difuprednate, enoxolone, fluazacort, flucloronide, flumethasone, flunisolide, fluocinolone acetonide, fluocinonide, flucloronide, flumethasone, flunisolide, fluocortin butyl, fluocortolone, fluorometholone, fluperolone acetate, flupreduisolone, fluraudrenolide, fluticasone propionate, hydrocortamate, hydrocortisone, meprednisone, methylpreduisolone, paramethasone, prednisolone, prednisolone 21-diethylaminoacetate, fluprednidene acetate, formocortal, loteprednol etabonate, medrysone, mometasone furoate, prednicarbate, prednisolone, prednisolone 25-diethyiaminoacetate, prednisolone sodium phosphate, prednisone, prednival, prednylidene, triamcinolone, triamcinolone acetonide, triamcinolone benetonide, and triamcinolone hexacetonide, and analogs, derivatives, pharmaceutically acceptable salts, esters, prodrugs, codrugs, and protected forms thereof. Suitable steroids include halcinonide, halbetasol propionate, halometasone, halopredone acetate, isoflupredone, loteprednol etabonate, niazipredone, rimexoione, and tixocortol, and analogs, derivatives, pharmaceutically acceptable salts, esters, prodrugs, co-drugs, and protected forms thereof.

In various embodiments, the steroid may be administered in an effective amount of at least about 10 mg to about 1500 mg. In various embodiments, the steroid may be administered in an effective amount of at least about 10, 25, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075 1100, 1125, 1150, 1175, 1200, 1225, 1250, 1275, 1300, 1325, 1350, 1375, 1400, 1425, 1450, 1475, and 1500 mg, or any range between any two of these amounts including from about 125 mg to about 1000 mg once daily. In some embodiments, the steroid may be administered at a dose of at least about 125 mg to about 1000 mg once daily for 3 days followed by a suitable tapering regimen as per clinical judgment.

In some forms, the composition will comprise each of the ingredients in a single administration form, such as a pill, tablet, capsule, oral solution, or any of the forms described herein. In other forms, the composition will comprise a kit comprising each of the individual ingredients, together with instructions for administering each ingredient. In some forms of the kit, certain ingredients will already be combined such that one, two, three, four, or more of the components or ingredients of the composition are in a single administration form as described herein.

This written description uses examples to disclose the subject matter herein, including the best mode, and also to enable any person skilled in the art to practice the subject matter disclosed herein, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A systemic pharmaceutical composition for the treatment of cytokine storm syndrome (CSS) comprising an effective amount of at least two mTor inhibitors, wherein the at least two mTor inhibitors comprise about 0.5 mg to about 40 mg of a non-antibiotic macrolide and about 250 mg to about 3000 mg of a biguanide antihyperglycemic agent.

2. The pharmaceutical composition of claim 1, further comprising at least one additional mTor inhibitor selected from the group consisting of: an omega-3 fatty acid derivative, resveratrol, and any combination thereof.

3. The pharmaceutical composition of claim 1, wherein the non-antibiotic macrolide is selected from the group consisting of rapamycin and a rapamycin derivative.

4. The pharmaceutical composition of claim 1, wherein the biguanide antihyperglycemic agent is metformin.

5. The pharmaceutical composition of claim 2, wherein the omega-3 fatty acid derivative is icosapent ethyl.

6. The pharmaceutical composition of claim 1, wherein the composition further comprises an effective amount of a steroid.

7. A method of treating, reducing the incidence of, or reducing the severity of CSS comprising administering an effective amount of the pharmaceutical composition of claim 1.

8. The method of claim 7, wherein the pharmaceutical composition of claim 1 further comprises at least one additional mTor inhibitor selected from the group consisting of: an omega-3 fatty acid derivative, resveratrol, and any combinations thereof.

9. The method of claim 7, wherein the non-antibiotic macrolide is selected from the group consisting of rapamycin and a rapamycin derivative.

10. The method of claim 7, wherein the biguanide antihyperglycemic agent is metformin.

11. The method of claim 8, wherein the at least one omega-3 fatty acid derivative is icosapent ethyl.

12. The method of claim 8, wherein the method comprises administering at least about 0.5 mg to about 40 mg of the non-antibiotic macrolide.

13. The method of claim 7, wherein the method further comprises administering an effective amount of a steroid.

14. A method of treating, or reducing the incidence of, or reducing the severity of acute respiratory distress syndrome comprising administering an effective amount of the pharmaceutical composition of claim 1.

15. The method of claim 14, wherein the pharmaceutical composition of claim 1 further comprises at least one additional mTor inhibitor selected from the group consisting of: an omega-3 fatty acid derivative, resveratrol, and any combination thereof.

16. The method of claim 14, wherein the non-antibiotic macrolide is selected from the group consisting of rapamycin and a rapamycin derivative.

17. The method of claim 14, wherein the biguanide antihyperglycemic agent is metformin.

18. The method of claim 15, wherein the at least one omega-3 fatty acid derivative is icosapent ethyl.

19. The method of claim 14, wherein the method further comprises administering an effective amount of a steroid.

20. A method of preventing multiple organ failure in a patient with CSS comprising administering an effective amount of the pharmaceutical composition of claim 1.
